(19) United States
(12) Patent Application Publication
(10) Pub. No.: US 2024/0058283 A1
(43) Pub. Date: Feb. 22, 2024

(54) THE MODE OF ACTION OF N-MeDCPA ON TRPC CHANNELS

(71) Applicants: West Virginia University Board of Governors on Behalf of West Virginia University, Morgantown, WV (US); Temple University-Of the Commonwealth System of Higher Education, Philadelphia, PA (US); University of Pittsburgh-Of the Commonwealth System of Higher Education, Pittsburgh, PA (US); The United States Government as represented by the Department of Veterans Affairs, Washington, DC (US)

(72) Inventors: John B. Barnett, Morgantown, WV (US); Werner Geldenhuys, Morgantown, WV (US); Jonathan Soboloff, Philadelphia, PA (US); Harry C. Blair, Pittsburgh, PA (US)

(21) Appl. No.: 18/336,712

(22) Filed: Jun. 16, 2023

Related U.S. Application Data
(60) Provisional application No. 63/366,585, filed on Jun. 17, 2022.

(51) Int. Cl. A61K 31/167

(52) U.S. Cl. CPC A61K 31/167

(57) ABSTRACT

A method of restoring the balance of osteoclast to osteoblast activity in a patient having rheumatoid arthritis comprising administering to a patient a therapeutically effective amount of the compound N-(3,4-dichlorophenyl)-N-methylpropanamide is provided. A method for treating arthritis-induced bone erosion in a patient comprising administering to a patient a therapeutically effective amount of the compound N-(3,4-dichlorophenyl)-N-methylpropanamide is provided. The compound N-(3,4-dichlorophenyl)-N-methylpropanamide is administered in a pharmaceutically acceptable composition to said patient. A mode of action of N-(3,4-dichlorophenyl)-N-methyl propanamide comprising targeting transient receptor potential channels is provided.

### CROSS-REFERENCE TO RELATED APPLICATION

This utility non-provisional patent application claims the benefit of priority to U.S. Provisional Patent Application Ser. No. 63/366,585, filed Jun. 17, 2022. The entire contents of U.S. Provisional Patent Application Ser. No. 63/366,585 is incorporated by reference into this utility non-provisional patent application as if fully rewritten herein.

### STATEMENT REGARDING FEDERALLY SPONSORED RESEARCH OR DEVELOPMENT

This invention was made with government support under Grant Nos. RO1 AR076146, RO1 DK119280, and R42 AR074812, awarded by the National Institute of Health, and Grant No. 2101 BX002490-06A1 awarded by the Department of Veterans Affairs. The government has certain rights in the invention.

### BACKGROUND OF THE INVENTION

### 1. Field of the Invention

This invention provides the targeting of cation channels called transient receptor potential channels (“TRPC”) for control and treatment of arthritis-induced bone erosion. In certain embodiments, this invention provides a method and mode of action of N-methyl-DCPA (i.e. N-MeDCPA; N-(3,4-dichlorophenyl)-N-methyl propanamide; ELP-004) on TRPC channels. A method of restoring the balance of osteoclast to osteoblast activity in a patient having rheumatoid arthritis is provided. A method for controlling or reducing arthritis-induced bone erosion is set forth.

### 2. Background Art

Inflammatory arthritis often requires treatments with serious side effects. Later in arthritis, bone erosion is a major problem that causes severe pain and debilitation. There is no small molecule drug available to specifically treat arthritis bone erosion. We show that osteoclast maturation is suppressed by blocking specific ion channels. An ion-channel antagonist, ELP-004, suppressed osteoclast maturation and strongly suppressed bone erosion in collagen-induced arthritis (CIA) in mice, even after symptoms of arthritis were measurable. In previous studies, we had focused on store-operated Ca²⁺ entry as the target of ELP-004, since Orail is required for osteoclast differentiation and ELP-004 blocks RANKL-induced osteoclast differentiation and SOCE at similar concentrations. However, ELP-004 blocks bone erosion in vivo at a remarkable 1000-fold higher efficacy, leading us to question the identity of the critical ion channel. To address this question, we performed a series of ELP-004 dose responses on HEK293 cells overexpressing different combinations of STIM/Orai, TRPC channels or CaV1.2. Remarkably, all TRPC channels were highly sensitive to ELP-004, leading us to consider the possibility that TRPC channels are the true target in arthritis-induced bone erosion. We are currently assessing the possibility that ELP-004 may inhibit osteoclastogenesis with higher efficacy when driven by inflammatory cytokines, both in vitro and in vivo. If so, these investigations will establish TRPC channels as critical mediators of arthritis-induced bone erosion and provide key insight into the true target of ELP-004, a patented drug currently in pre-clinical testing as a potential therapeutic.

Acute arthritis occurs in solitary sites after trauma or can be caused by infections, such as in Lyme disease, in adults or children. Usually, a cause is not identified¹. Rheumatoid arthritis (RA) typically has an inflammatory pattern indistinguishable from acute arthritis, but it has a chronic relapsing course leading to the widespread destruction that involves many joints and usually requires lifelong therapy. While there are infectious causes and genetic predilections, typically RA also is idiopathic. Treatments for inflammatory arthritis include anti-metabolites, steroids, and TNF-α-blocking molecules that cause dangerous or debilitating side effects².

### SUMMARY OF THE INVENTION

In one embodiment of this invention, a method of restoring the balance of osteoclast to osteoblast activity in a patient having rheumatoid arthritis is provided comprising administering to a patient a therapeutically effective amount of a compound N-(3,4-dichlorophenyl)-N-methylpropanamide for restoring the balance of osteoclast to osteoblast activity in said patient. This method includes wherein said compound N-(3,4-dichlorophenyl)-N-methylpropanamide is administered in a pharmaceutically acceptable composition to said patient. This method includes wherein said pharmaceutically acceptable composition is administered to said patient via the oral route of administration.

In another embodiment of this invention, a method for treating arthritis-induced bone erosion in a patient is provided comprising administering to a patient a therapeutically effective amount of a compound N-(3,4-dichlorophenyl)-N-methylpropanamide for treating arthritis-induced bone erosion in said patient. This method includes wherein said compound N-(3,4-dichlorophenyl)-N-methylpropanamide is administered in a pharmaceutically acceptable composition to said patient. This method includes wherein said pharmaceutically acceptable composition is administered to said patient via the oral route of administration.

In yet another embodiment of this invention, a mode of action of N-methyl-DCPA (N-(3,4-dichlorophenyl)-N-methylpropanamide) is provided comprising targeting a transient receptor potential channel. This method comprises targeting a transient receptor potential channel (i.e. a TRPC) by administering to a patient a therapeutically effective amount of N-(3,4-dichlorophenyl)N-methylpropanamide to said patient. This method includes wherein said compound N-(3,4-dichlorophenyl)-N-methylpropanamide is administered in a pharmaceutically acceptable composition to said patient. This method includes wherein said pharmaceutically acceptable composition is administered to said patient via the oral route of administration. The transient receptor potential channel is selected from the group consisting of a TRPC3 channel, a TRPC6 channel, and a TRPC7 channel.

In another embodiment of this invention, a method of directly targeting osteoclast cells that remove old bone in a patient is provided comprising administering to a patient a therapeutically effective amount of a compound N-(3,4-dichlorophenyl)-N-methylpropanamide for directly targeting osteoclasts and restoring normal bone health in said patient. This method incudes restoring normal bone health in a patient having a disease. This method includes wherein said disease is rheumatoid arthritis. This method includes wherein said compound N-(3,4-dichlorophenyl)-N-methylpropanamide is administered in a pharmaceutically acceptable composition to said patient. This method includes wherein said pharmaceutically acceptable composition is administered to said patient via the oral route of administration.

### BRIEF DESCRIPTION OF THE DRAWINGS

FIG. 1A shows that ELP-004 inhibits DAG-induced TRPC3 activation. FIG. 1A shows representative traces of 1-Oleoyl-2-acetyl-sn-glycerol (OAG)-induced Ca²⁺ entry in Fura2-loaded HEK293 cells overexpressing TRPC3 treated with ELP-004 at the indicated concentrations.

FIG. 1B shows that ELP-004 inhibits DAG-induced TRPC6 activation. FIG. 1B shows representative traces of 1-Oleoyl-2-acetyl-sn-glycerol (OAG)-induced Ca²⁺ entry in Fura2-loaded HEK293 cells overexpressing TRPC6 treated with ELP-004 at the indicated concentrations.

FIG. 1C shows that ELP-004 inhibits DAG-induced TRPC7 activation. FIG. 1C shows representative traces of 1-Oleoyl-2-acetyl-sn-glycerol (OAG)-induced Ca²⁺ entry in Fura2-loaded HEK293 cells overexpressing TRPC7 treated with ELP-004 at the indicated concentrations.

FIG. 1D shows Ca²⁺ entry was quantified under each condition and that dose dependence was determined by on-linear regression (n≥5).

FIG. 2A shows high efficacy inhibition of PLC-mediated TRPC activation by ELP-004. Representative traces of carbachol (Cch)-induced Sr²⁺ entry in Fura2-loaded HEK293 cells overexpressing TRPC4.

FIG. 2B shows high efficacy inhibition of PLC-mediated TRPC activation by ELP-004. Representative traces of carbachol (Cch)-induced Sr²⁺ entry in Fura2-loaded HEK293 cells overexpressing TRPC5.

FIG. 2C shows Sr²⁺ entry was quantified under each condition and dose dependence was determined by non-linear regression (n≥5).

FIG. 2D shows representative traces of Cch-induced Sr²⁺ entry in Fura2-loaded HEK293 cells overexpressing treated with ELP-004 at the indicated concentrations.

FIG. 2E shows Sr²⁺ entry was quantified under each condition and dose dependence was determined by non-linear regression (n≥5).

FIG. 3 shows metabolite formation and the structure of ELP-004.

FIG. 4 shows the IC₅₀ (μM) of TRPC4 and TRPC5 and that TRPC5 does not reach the threshold of >50% inhibition.

FIG. 5 shows that ELP-004 blocks TRPC7 channels with an IC₅₀ of 9 nM.

FIG. 6 shows that ELP-004 blocks osteoclast resorption.

### DETAILED DESCRIPTION OF THE INVENTION

We have extensively investigated inhibitors of the store-operated Ca²⁺ entry (SOCE) pathway as a target for control of bone erosion, particularly in the context of arthritis. Hence, suppressing SOCE via siRNA targeting Orail (the pore-forming unit of the store-operated Ca²⁺ channel) or the antagonist, 3,4-dichloropropionaniline (DCPA) reduced the formation of bone degrading osteoclasts in vitro³. Further, Orail-KO mice exhibit a significant loss of multinuclear osteoclasts and defects in bone degradation^{4,5}. On this basis, we have assessed the possibility that DCPA and/or DCPA analogs might be effective inhibitors of bone erosion in the context of arthritis⁶. While this has proven to the case (data not shown), this investigation has opened new questions regarding the true identity of the DCPA target. Hence, neither DCPA nor its analogs block osteoclast differentiation at concentrations below 100 μM³, yet serum levels of these agents fail to go above 100 nM in vivo. As such, we have assessed the possibility that DCPA and its analogs may have a different target, especially within the context of arthritis.

The current investigation focuses primarily on members of the canonical transient receptor potential (TRPC) family of non-selective ion channels. Although TRPC channels have been extensively investigated as potential store-operated Ca²⁺ channels⁷⁻⁹, they are primarily activated by receptors that activate phospholipase C (PLC). Phospholipase C is a widely expressed enzyme activated by a large range of G protein-coupled receptors (GPCRs) and tyrosine kinase receptors (TKRs). Upon activation, PLC degrades phosphatidylinositol into diacylgycerol (DAG) and IP₃; DAG directly activates TRPC3, 6 and 7 while loss of phosphatidylinositol is thought to be drive activity of TRPC1, 4 and 5; TRPC2 is a pseudogene in humans. As discussed below, we find that the DCPA analog N-MeDCPA inhibits TRPC channels with a 2 to 5 order of magnitude higher efficacy than SOCE, making TRPC channels the most likely candidate targets for N-MeDCPA-mediated blockade of bone erosion associated with arthritis.

N-MeDCPA (i.e. ELP-004; N-(3,4-dichlorophenyl)-N-methylpropanamide) is a haloanilide compound of Formula I:

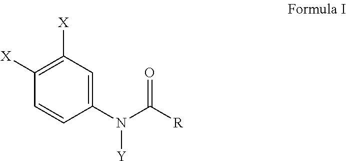

wherein X is chlorine, Y is a methyl group, and R is an ethyl group. N-MeDCPA is described in U.S. Pat. No. 10,682,320, and is incorporated by reference herein.

Results

Development and safety profile of N-MeDCPA as a lead compound.

ELP-004 (N-MeDCPA) was specifically developed to preclude metabolizing to two toxic metabolites, N—OH-dichloroaniline and 6-OH-dichloroaniline. In vitro assays for toxicity caused by ELP-004 using Jurkat cells show 50% toxicity at 800 mM and <2% toxicity at 200 mM. The Contract Research Organization Illinois Institute of Toxicology Research Institute (IITRI) performed 1× escalating toxicity in rats and determined that the oral LD₅₀ was >2.5 g/kg body weight. Excellent in vivo efficacy to prevent bone erosion associated with arthritis using two mouse models, was achieved using 150 mM intraperitoneally in an N-MeDCPA:nanoparticle preparation. Thus, ELP-004 has high potential for use as a drug to prevent or reduce bone erosion.

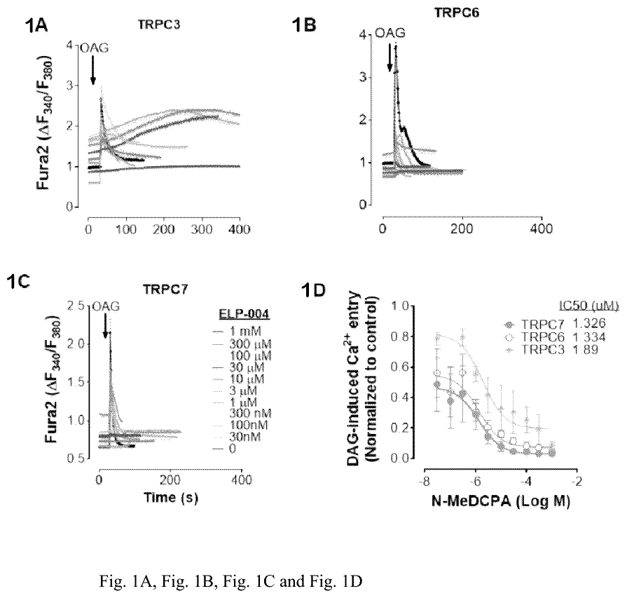

Inhibition of DAG-mediated TRPC activation. To determine if ELP-004 (N-MeDCPA) inhibits DAG-induced TRPC activation, HEK293 cells expressing TRPC3, TRPC6 or TRPC7 were loaded with Fura2 and incubated with ELP-004 (0 to 1 mM) for 10 minutes prior to challenging with 1-Oleoyl-2-acetyl-sn-glycerol (OAG), a membrane permeable form of diacylglycerol. OAG stimulated rapid Ca²⁺ entry in untreated TRPC-expressing cells (FIG. 1A-C), inhibition of OAG-induced Ca²⁺ entry by ELP-004 was observed with an IC50 of 1 to 2 μM (FIG. 1D). As this represents a 2 order of magnitude improvement in efficacy, these data provide proof-of-principle that the true target of ELP-004 may be TRPC and not store-operated Ca²⁺ channels.

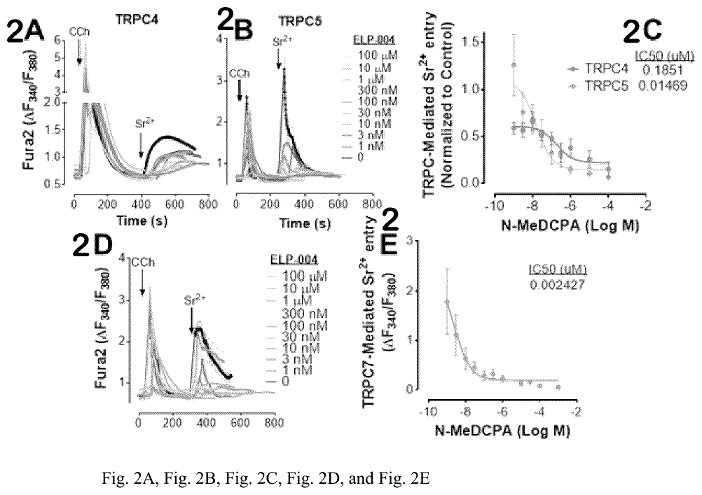

Inhibition of GPCR-mediated TRPC activation. Since TRPC4 and TRPC5 are not activated by DAG, we utilized carbachol (Cch)-induced Sr²⁺ entry to determine their sensitivity to ELP-004. Since Cch drives ER Ca²⁺ release, Cch-induced Ca²⁺ entry includes both store-operated and TRPC-mediated components. To specifically measure ion entry through TRPC channels, we took advantage of the lack of ion selectivity of TRPC channel and substituted Sr²⁺ for Ca²⁺ as previously described⁷. As expected, Cch stimulated rapid ER Ca²⁺ release immediately upon addition in both TRPC4- and TRPC5-expressing cells (FIG. 2A,B). The subsequent addition of Sr²⁺ (3 mM) lead to a significant increase in Fura ratio with a substantially larger response in TRPC5-expressing than TRPC4-expressing cells. The addition of ELP-004 (0 to 100 μM) dose dependently inhibited TRPC-mediated Sr²⁺ entry, with 1 to 2 orders of magnitude (TRPC5>TRPC4) higher efficacy than DAG-mediated TRPC activation (FIG. 2C). Since different assays were used on different ion channels, whether or not TRPC4 and TRPC5 are more sensitive to ELP-004 than TRPC3, TRPC6 and TRPC7 or if ELP-004 inhibits DAG-independent TRPC activation with higher efficacy than DAG-induced TRPC activation is not clear. Therefore, we assessed inhibition of TRPC7 by ELP-004 using the Cch Sr²⁺ assay (FIG. 2D, E). Remarkably, TRPC7 exhibited a 3 order of magnitude increase in sensitivity to ELP-004 using this assay, suggesting that receptor-mediated TRPC activation is more sensitive to ELP-004 than direct DAG-mediated TRPC activation.

Since receptor-mediated TRPC activation occurred with a much higher sensitivity than DAG-mediated TRPC activation, these data provide insight into fundamental differences in these 2 different modes of TRPC activation. Future investigations focused on defining these differences may provide new insights into precisely how PLC-coupled receptors activate TRPC channels, a question that remains largely unclear.

The other major question remaining is to define the context whereby TRPC channels would contribute to osteoclastogenesis. While physiological osteoclastogenesis is primarily driven by RANKL, in arthritis, local increases in cytokines are believed to drive osteoclastogenesis; while RANKL is not PLC-coupled, many cytokines are. As such, ongoing investigations are directed at determining if TRPC channels may contribute to arthritis-induced osteoclastogenesis driven by PLC-coupled cytokines.

Materials and Methods

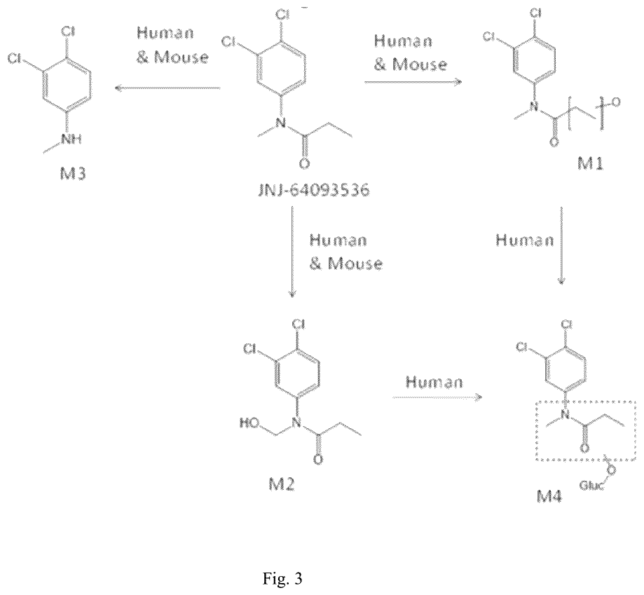

Drug Synthesis—ELP-004 (N-MeDCPA) was synthesized as described in U.S. patent Ser. No. 10/682,320. FIG. 3 shows the metabolite formation. Metabolites formed after incubations using either cofactors-fortified mouse or human liver S9 (mouse liver S9; human liver S9) at 37 degrees centigrade for 1 hour. The reaction was quenched by ultracentrifugation. The supernatant was analyzed using LC-MS/MS analysis using Accela LC (Thermo Scientific, Inc., San Jose, CA) coupled to an LTQ LX or to LTQ Orbitrap XL. The proposed metabolic pathway of ELP-004 in human and mouse liver S9 is shown in FIG. 3.

Cell Culture—HEK293 cells were grown in DMEM with 4.5 g/L glucose supplemented with 10% FBS and 1% Gentamycin (Full DMEM media; Thermo-Fisher Scientific, Waltham, MA) at 37° C. and 5% CO₂. For measurements of TRPC4, TRPC5 or TRPC7 activity, cells were transiently transfected with over-expression plasmids and incubated for 2 days prior to loading with Fura2AM. HEK293 cells stably expressing either TRPC3 or TRPC6 were a generous gift from Dr. Donald L. Gill (Penn State University).

TRPC activity assay—Cells grown on glass coverslips were incubated in a cation-safe buffer (107 mM NaCl, 7.2 mM KCl, 1.2 mM MgCl₂, 11.5 mM Glucose, 20 mM HEPES-NaOH, 1 mM CaCl₂), pH 7.2) and loaded with Fura2-acetoxymethylester (Fura2-AM; 2 μM) for 30 min at 24° C. as previously described^{10,11}. Cells were washed and allowed to de-esterify for a minimum of 30 min at 24° C. Ca²⁺/Sr²⁺ measurements were taken using a Leica DMI 6000B fluorescence microscope controlled by Slidebook software (Intelligent Imaging Innovations, Denver, CO). Fluorescence emission at 505 nm was monitored in response to excitation at alternating 340 nm and 380 nm wavelengths at a frequency of 0.67 Hz; intracellular Ca²⁺ measurements are shown at 340/380 nm ratios obtained from groups of 35-45 single cells.

ELP-004 blocks TNF-α-induced osteoclast differentiation. Mouse splenic monocytes were induced to differentiate into osteoclasts. Results were obtained of osteoclast cells after 21 days of culture with RANKL+m-CSF alone or with the addition of 100 nM ELP-004. ELP-004 inhibited development of multinucleated osteoclasts. Thus, ELP-004 inhibits osteoclastogenesis.

FIG. 3 shows the metabolic pathway of ELP-004 I human and mouse liver S9. ELP-004 is N-(3,4-dichlorophenyl)-N-methylpropanamide. The core structure of ELP-004 is an aniline compound. Aniline compounds, generally, have a high risk of forming reactive metabolites. ELP-004 was specifically designed to preclude formation of reactive metabolites. Methylation of the amide nitrogen improves metabolic stability. No reactive or toxic compounds were formed, and some metabolites became glucuronidated and thus rapidly removed by the kidney.

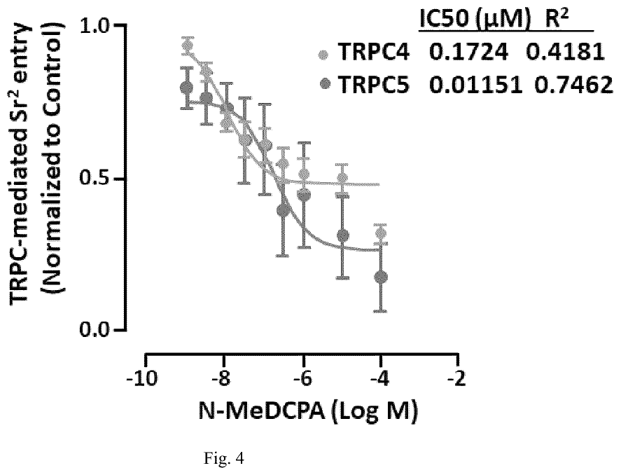

FIG. 4 shows that TRPC4 and TRPC5 Overexpressing (O/E) cells were incubated in fura and Ca²⁺ solution. The assay was done in the presence of Ca²⁺. OAG (1-Oleoyl-2-acetyl-sn-glycerol) is a cell permeable DAG analog and directly activates these TRPC channels (i.e. TRPC4 and TRPC5). TRPC4 IC₅₀=172 nM and TRPC5 IC₅₀=11 nM. TRPC5 does not reach the threshold of >50% inhibition (N=4).

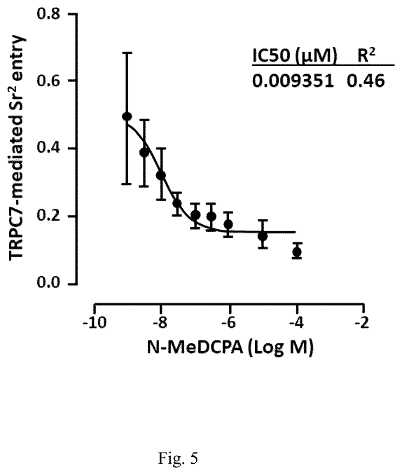

FIG. 5 shows that ELP-004 blocks TRPC7 channels with an IC₅₀ of 9 nM. TRPC Overexpressing (O/E) cells were incubated in fura and Ca²⁺ solution. The assay was done in the presence of Ca²⁺. OAG (1-Oleoyl-2-acetyl-sn-glycerol) is a cell permeable DAG analog and directly activates TRPC7 channels.

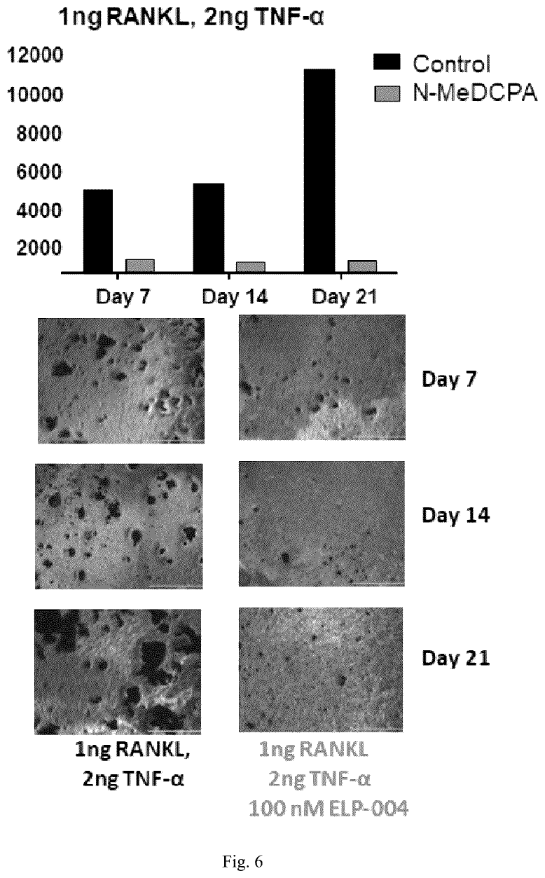

FIG. 6 shows that ELP-004 blocks osteoclast resorption. Mouse splenic monocytes were induced to differentiate by the addition of 1 ng/ml RANKL, 2 ng/ml TNFα and 50 ng/ml m-CSF. The panels of FIG. 6 show a representative photomicrograph of cells after 7, 14, or 21 days of culture on Corning osteo-assay surface plates with RANKL+mCSF+TNFα or with the addition of 100 nM ELP-004. ELP-004 inhibited pit formation that was induced by mCSF and TNFα at concentrations that are congruent with in vivo does of ELP-004 that inhibit bone erosion.

ELP-004 was evaluated for its ability to cross the blood brain barrier. ELP-004 does not cross the blood brain barrier. Further, ELP-004 does not cause problems with dopamine transport. Other medications, such as for example but not limited to bupropion are known to cause problems due to dopamine transporter binding. The half-life of ELP-004 when administered per os (i.e. by mouth) indicates that ELP-004 is essentially eliminated within 4 hour, given the short half-life and the relative quick time to Cmax the dopaminergic side effects would occur within 1-2 hours of administration of ELP-004. Thus, as ELP-004 does not cross the blood brain barrier, this indicates that the dopamine transporter binding is not concerning regarding ELP-004. ELP-004 has no deleterious effect on induction of CYP enzymes.

### REFERENCES

1. Till, S. H. & Snaith, M. L. Assessment, investigation, and management of acute monoarthritis. Journal of accident & emergency medicine 16, 355-361 (1999). PMCID:1347058. <http://www.ncbi.nlm.nih.gov/pubmed/10505918>
2. Gaffo, A., Saag, K. G. & Curtis, J. R. Treatment of rheumatoid arthritis. American journal of health-system pharmacy: AJHP: official journal of the American Society of Health-System Pharmacists 63, 2451-2465 (2006). <http://www.ncbi.nlm.nih.gov/pubmed/17158693>
3. Zhou, Y., Lewis, T. L., Robinson, L. J., Brundage, K. M., Schafer, R., Martin, K. H., Blair, H. C., Soboloff, J. & Barnett, J. B. The role of calcium release activated calcium channels in osteoclast differentiation. Journal of cellular physiology 226, 1082-1089 (2011). PMCID:4020518. <http://www.ncbi.nlm.nih.gov/pubmed/20839232>
4. Robinson, L. J., Mancarella, S., Songsawad, D., Tourkova, I. L., Barnett, J. B., Gill, D. L., Soboloff, J. & Blair, H. C. Gene disruption of the calcium channel Orail results in inhibition of osteoclast and osteoblast differentiation and impairs skeletal development. Laboratory investigation 92, 1071-1083 (2012). PMCID:3387291. <http://www.ncbi.nlm.nih.gov/pubmed/10505918>
5. Robinson, L. J., Soboloff, J., Tourkova, I. L., Larrouture, Q. C., Witt, M. R., Gross, S., Hooper, R., Samakai, E., Worley, P. F., Barnett, J. B. & Blair, H. C. The function of the calcium channel Orail in osteoclast development. FASEB J 35, e21653 (2021). <http://www.ncbi.nlm.nih.gov/pubmed/10505918>
6. Blair, H. C., Soboloff, J., Robinson, L. J., Tourkova, I. L., Larrouture, Q. C., Witt, M. R., Holaskova, I., Schafer, R., Elliott, M., Hirsch, R. & Barnett, J. B. Suppression of arthritis-induced bone erosion by a CRAC channel antagonist. RMD Open 2, e000093 (2016). PMCID:4716559. <http://www.ncbi.nlm.nih.gov/pubmed/26816750>
7. He, L. P., Hewavitharana, T., Soboloff, J., Spassova, M. A. & Gill, D. L. A functional link between store-operated and TRPC channels revealed by the 3,5-bis(trifluoromethyl)pyrazole derivative, BTP2. J Biol Chem 280, 10997-11006 (2005). <http://www.ncbi.nlm.nih.gov/pubmed/15647288>
8. Ong, H. L., Cheng, K. T., Liu, X., Bandyopadhyay, B. C., Paria, B. C., Soboloff, J., Pani, B., Gwack, Y., Srikanth, S., Singh, B. B., Gill, D. & Ambudkar, I. S. Dynamic assembly of TRPC1-STIM1-Orail ternary complex is involved in store-operated calcium influx—Evidence for similarities in store-operated and calcium release-activated calcium channel components. J. Biol. Chem. 282, 9105-9116 (2007). <<Go to SI>://WOS:000245780300058>
9. Soboloff, J., Rothberg, B. S., Madesh, M. & Gill, D. L. STIM proteins: dynamic calcium signal transducers. Nat Rev Mol Cell Biol 13, 549-565 (2012). PMCID:3458427. <http://www.ncbi.nlm.nih.gov/pubmed/22914293>
10. Hooper, R., Zhang, X., Webster, M., Go, C., Kedra, J., Marchbank, K., Gill, D. L., Weeraratna, A. T., Trebak, M. & Soboloff, J. Novel Protein Kinase C-Mediated Control of Orail Function in Invasive Melanoma. Mol Cell Biol 35, 2790-2798 (2015). PMCID:4508319. <http://www.ncbi.nlm.nih.gov/pubmed/26055321>
11. Go, C. K., Hooper, R., Aronson, M. R., Schultz, B., Cangoz, T., Nemani, N., Zhang, Y., Madesh, M. & Soboloff, J. The Ca(2+) export pump PMCA clears near-membrane Ca(2+) to facilitate store-operated Ca(2+) entry and NFAT activation. Sci Signal 12 (2019). PMCID:PMC6800072. <http://www.ncbi.nlm.nih.gov/pubmed/31594854>

These terms and specifications, including examples, serve to describe the invention by example and not to limit the invention. Whereas particular embodiments of this invention have been described for purposes of illustration, it will be evident to those persons skilled in the art that numerous variations of the details of the present invention may be made without departing from the invention as defined herein and in the appended claims.

What is claimed is:

1. A method of restoring the balance of osteoclast to osteoblast activity in a patient having rheumatoid arthritis comprising administering to a patient a therapeutically effective amount of a compound N-(3,4-dichlorophenyl)-N-methylpropanamide for restoring the balance of osteoclast to osteoblast activity in said patient.

2. The method of claim 1 wherein said compound N-(3,4-dichlorophenyl)-N-methylpropanamide is administered in a pharmaceutically acceptable composition to said patient.

3. A method of claim 2 wherein said pharmaceutically acceptable composition is administered to said patient via the oral route of administration.

4. A method for treating arthritis-induced bone erosion in a patient comprising administering to a patient a therapeutically effective amount of a compound N-(3,4-dichlorophenyl)-N-methylpropanamide for treating arthritis-induced bone erosion in said patient.

5. The method of claim 4 wherein said compound N-(3,4-dichlorophenyl)-N-methylpropanamide is administered in a pharmaceutically acceptable composition to said patient.

6. A method of claim 5 wherein said pharmaceutically acceptable composition is administered to said patient via the oral route of administration.

7. A mode of action of N-methyl-DCPA (i.e. N-(3,4-dichlorophenyl)-N-methylpropanamide) comprising targeting a transient receptor potential channel.

8. The mode of action of claim 7 including wherein said transient receptor potential channel is selected from the group consisting of a TRPC3 channel, a TRPC6 channel, and a TRPC7 channel.

9. A method of targeting a transient receptor potential channel comprising administering to a patient a therapeutically effective amount of N-(3,4-dichlorophenyl)-N-methylpropanamide to said patient for targeting said transient receptor potential channel.

10. The method of claim 9 including wherein said compound N-(3,4-dichlorophenyl)-N-methylpropanamide is administered in a pharmaceutically acceptable composition to said patient.

11. The method of claim 10 including wherein said pharmaceutically acceptable composition is administered to said patient via the oral route of administration.

12. The method of claim 9 including wherein said transient receptor potential channel is selected from the group consisting of a TRPC3 channel, a TRPC6 channel, and a TRPC7 channel.

13. A method of directly targeting osteoclast cells that remove old bone in a patient comprising administering to a patient a therapeutically effective amount of a compound N-(3,4-dichlorophenyl)-N-methylpropanamide for directly targeting osteoclasts and restoring normal bone health in said patient.

14. The method of claim 13 including restoring normal bone health in a patient having a disease.

15. The method of claim 14 including wherein said disease is rheumatoid arthritis.

16. The method of claim 13 including wherein said compound N-(3,4-dichlorophenyl)-N-methylpropanamide is administered in a pharmaceutically acceptable composition to said patient.

17. The method of claim 16 wherein said pharmaceutically acceptable composition is administered to said patient via the oral route of administration.
